(19) United States
(12) Patent Application Publication
(10) Pub. No.: US 2005/0255041 A1
(43) Pub. Date: Nov. 17, 2005

(54) Cancerous disease modifying antibodies

(75) Inventors: David S. F. Young, Toronto (CA); Susan E. Hahn, Toronto (CA); Helen P. Findlay, Toronto (CA)

Correspondence Address: MCHALE & SLAVIN, P.A., 2855 PGA BLVD, PALM BEACH GARDENS, FL, 33410, US

(73) Assignee: Arius Research, Inc., Toronto (CA)

(21) Appl. No.: 10/846,266

(22) Filed: May 13, 2004

(51) Int. Cl. A61K 51/00; A61K 39/395

(52) U.S. Cl. 424/1.49; 424/155.1; 424/178.1

(57) ABSTRACT

The present invention relates to a method for producing patient cancerous disease modifying antibodies using a novel paradigm of screening. By segregating the anti-cancer antibodies using cancer cell cytotoxicity as an end point, the process makes possible the production of anti-cancer antibodies for therapeutic and diagnostic purposes. The antibodies can be used in aid of staging and diagnosis of a cancer, and can be used to treat primary tumors and tumor metastases. The anti-cancer antibodies can be conjugated to toxins, enzymes, radioactive compounds, and hematogenous cells.

### FIELD OF THE INVENTION

This invention relates to the isolation and production of cancerous disease modifying antibodies (CDMAB) and to the use of these CDMAB in therapeutic and diagnostic processes, optionally in combination with one or more chemotherapeutic agents. The invention further relates to binding assays which utilize the CDMAB of the instant invention.

### BACKGROUND OF THE INVENTION

Each individual who presents with cancer is unique and has a cancer that is as different from other cancers as that person's identity. Despite this, current therapy treats all patients with the same type of cancer, at the same stage, in the same way. At least 30 percent of these patients will fail the first line therapy, thus leading to further rounds of treatment and the increased probability of treatment failure, metastases, and ultimately, death. A superior approach to treatment would be the customization of therapy for the particular individual. The only current therapy that lends itself to customization is surgery. Chemotherapy and radiation treatment cannot be tailored to the patient, and in most cases, surgery by itself is inadequate for producing cures.

With the advent of monoclonal antibodies, the possibility of developing methods for customized therapy became more realistic since each antibody can be directed to a single epitope. Furthermore, it is possible to produce a combination of antibodies that are directed to the constellation of epitopes that uniquely define a particular individual's tumor.

Having recognized that a significant difference between cancerous and normal cells is that cancerous cells contain antigens that are specific to transformed cells, the scientific community has long held that monoclonal antibodies can be designed to specifically target transformed cells by binding specifically to these cancer antigens. This has given rise to the belief that monoclonal antibodies can serve as “Magic Bullets” to eliminate cancer cells.

Monoclonal antibodies isolated in accordance with the teachings of the instantly disclosed invention have been shown to modify the cancerous disease process in a manner which is beneficial to the patient, for example by reducing the tumor burden, and will variously be referred to herein as cancerous disease modifying antibodies (CDMAB) or “anti-cancer” antibodies.

At the present time, the cancer patient usually has few options of treatment. The regimented approach to cancer therapy has produced improvements in global survival and morbidity rates. However, to the particular individual, these improved statistics do not necessarily correlate with an improvement in their personal situation.

Thus, if a methodology was put forth which enabled the practitioner to treat each tumor independently of other patients in the same cohort, this would permit the unique approach of tailoring therapy to just that one person. Such a course of therapy would, ideally, increase the rate of cures, and produce better outcomes, thereby satisfying a long-felt need.

Historically, the use of polyclonal antibodies has been used with limited success in the treatment of human cancers. Lymphomas and leukemias have been treated with human plasma, but there were few prolonged remission or responses. Furthermore, there was a lack of reproducibility and no additional benefit compared to chemotherapy. Solid tumors such as breast cancers, melanomas and renal cell carcinomas have also been treated with human blood, chimpanzee serum, human plasma and horse serum with correspondingly unpredictable and ineffective results.

There have been many clinical trials of monoclonal antibodies for solid tumors. In the 1980s there were at least 4 clinical trials for human breast cancer which produced only 1 responder from at least 47 patients using antibodies against specific antigens or based on tissue selectivity. It was not until 1998 that there was a successful clinical trial using a humanized anti-her 2 antibody in combination with cisplatin. In this trial 37 patients were accessed for responses of which about a quarter had a partial response rate and another half had minor or stable disease progression.

The clinical trials investigating colorectal cancer involve antibodies against both glycoprotein and glycolipid targets. Antibodies such as 17-1A, which has some specificity for adenocarcinomas, has undergone Phase 2 clinical trials in over 60 patients with only 1 patient having a partial response. In other trials, use of 17-1A produced only 1 complete response and 2 minor responses among 52 patients in protocols using additional cyclophosphamide. Other trials involving 17-1A yielded results that were similar. The use of a humanized murine monoclonal antibody initially approved for imaging also did not produce tumor regression. To date there has not been an antibody that has been effective for colorectal cancer. Likewise there have been equally poor results for lung, brain, ovarian, pancreatic, prostate, and stomach cancers. There has been some limited success in the use of anti-GD3 monoclonal antibodies for melanoma. Thus, it can be seen that despite successful small animal studies that are a prerequisite for human clinical trials, the antibodies that have been tested thus far, have been for the most part, ineffective.

Prior Patents:

U.S. Pat. No. 5,750,102 discloses a process wherein cells from a patient's tumor are transfected with MHC genes which may be cloned from cells or tissue from the patient. These transfected cells are then used to vaccinate the patient.

U.S. Pat. No. 4,861,581 discloses a process comprising the steps of obtaining monoclonal antibodies that are specific to an internal cellular component of neoplastic and normal cells of the mammal but not to external components, labeling the monoclonal antibody, contacting the labeled antibody with tissue of a mammal that has received therapy to kill neoplastic cells, and determining the effectiveness of therapy by measuring the binding of the labeled antibody to the internal cellular component of the degenerating neoplastic cells. In preparing antibodies directed to human intracellular antigens, the patentee recognizes that malignant cells represent a convenient source of such antigens.

U.S. Pat. No. 5,171,665 provides a novel antibody and method for its production. Specifically, the patent teaches formation of a monoclonal antibody which has the property of binding strongly to a protein antigen associated with human tumors, e.g. those of the colon and lung, while binding to normal cells to a much lesser degree.

U.S. Pat. No. 5,484,596 provides a method of cancer therapy comprising surgically removing tumor tissue from a human cancer patient, treating the tumor tissue to obtain tumor cells, irradiating the tumor cells to be viable but non-tumorigenic, and using these cells to prepare a vaccine for the patient capable of inhibiting recurrence of the primary tumor while simultaneously inhibiting metastases. The patent teaches the development of monoclonal antibodies which are reactive with surface antigens of tumor cells. As set forth at col. 4, lines 45 et seq., the patentees utilize autochthonous tumor cells in the development of monoclonal antibodies expressing active specific immunotherapy in human neoplasia.

U.S. Pat. No. 5,693,763 teaches a glycoprotein antigen characteristic of human carcinomas is not dependent upon the epithelial tissue of origin.

U.S. Pat. No. 5,783,186 is drawn to anti-Her2 antibodies which induce apoptosis in Her2 expressing cells, hybridoma cell lines producing the antibodies, methods of treating cancer using the antibodies and pharmaceutical compositions including said antibodies.

U.S. Pat. No. 5,849,876 describes new hybridoma cell lines for the production of monoclonal antibodies to mucin antigens purified from tumor and non-tumor tissue sources.

U.S. Pat. No. 5,869,268 is drawn to a method for generating a human lymphocyte producing an antibody specific to a desired antigen, a method for producing a monoclonal antibody, as well as monoclonal antibodies produced by the method. The patent is particularly drawn to the production of an anti-HD human monoclonal antibody useful for the diagnosis and treatment of cancers.

U.S. Pat. No. 5,869,045 relates to antibodies, antibody fragments, antibody conjugates and single chain immunotoxins reactive with human carcinoma cells. The mechanism by which these antibodies function is two-fold, in that the molecules are reactive with cell membrane antigens present on the surface of human carcinomas, and further in that the antibodies have the ability to internalize within the carcinoma cells, subsequent to binding, making them especially useful for forming antibody-drug and antibody-toxin conjugates. In their unmodified form the antibodies also manifest cytotoxic properties at specific concentrations.

U.S. Pat. No. 5,780,033 discloses the use of autoantibodies for tumor therapy and prophylaxis. However, this antibody is an anti-nuclear autoantibody from an aged mammal. In this case, the autoantibody is said to be one type of natural antibody found in the immune system. Because the autoantibody comes from “an aged mammal”, there is no requirement that the autoantibody actually comes from the patient being treated. In addition the patent discloses natural and monoclonal anti-nuclear autoantibody from an aged mammal, and a hybridoma cell line producing a monoclonal anti-nuclear autoantibody.

### SUMMARY OF THE INVENTION

The instant inventors have previously been awarded U.S. Pat. No. 6,180,357, entitled “Individualized Patient Specific Anti-Cancer Antibodies” directed to a process for selecting individually customized anti-cancer antibodies which are useful in treating a cancerous disease, the contents of which are herein incorporated by reference. For the purpose of this document, the terms “antibody” and “monoclonal antibody” (mAb) may be used interchangeably and refer to intact immunoglobulins produced by hybridomas (e.g. murine or human), immunoconjugates and, as appropriate, immunoglobulin fragments and recombinant proteins derived from immunoglobulins, such as chimeric and humanized immunoglobulins, F(ab′) and F(ab′)₂ fragments, single-chain antibodies, recombinant immunoglobulin variable regions (Fv)s, fusion proteins etc. For the purpose of this document, the term “tissue sample” is understood to mean at least one cell or an aggregate of cells obtained from a mammal. It is well recognized in the art that some amino acid sequence can be varied in a polypeptide without significant effect on the structure or function of the protein. In the molecular rearrangement of antibodies, modifications in the nucleic or amino acid sequence of the backbone region can generally be tolerated. These include, but are not limited to, substitutions (preferred are conservative substitutions), deletions or additions. Furthermore, it is within the purview of this invention to conjugate standard chemotherapeutic modalities, e.g. radionuclides, with the CDMAB of the instant invention, thereby focusing the use of said chemotherapeutics. The CDMAB can also be conjugated to toxins, cytotoxic moieties, enzymes e.g. biotin conjugated enzymes, or hematogenous cells, thereby forming antibody conjugates. Such conjugated moieties are illustrated herein as conjugated to the monoclonal antibody derived from the hybridoma cell line designated AR2A72.10.

This application utilizes the method for producing patient specific anti-cancer antibodies as taught in the '357 patent for isolating hybridoma cell lines which encode for cancerous disease modifying monoclonal antibodies. These antibodies can be made specifically for one tumor and thus make possible the customization of cancer therapy. Within the context of this application, anti-cancer antibodies having either cell-killing (cytotoxic) or cell-growth inhibiting (cytostatic) properties will hereafter be referred to as cytotoxic. These antibodies can be used in aid of staging and diagnosis of a cancer, and can be used to treat tumor metastases.

The prospect of individualized anti-cancer treatment will bring about a change in the way a patient is managed. A likely clinical scenario is that a tumor sample is obtained at the time of presentation, and banked. From this sample, the tumor can be typed from a panel of pre-existing cancerous disease modifying antibodies. The patient will be conventionally staged but the available antibodies can be of use in further staging the patient. The patient can be treated immediately with the existing antibodies and/or a panel of antibodies specific to the tumor can be produced either using the methods outlined herein or through the use of phage display libraries in conjunction with the screening methods herein disclosed. All the antibodies generated will be added to the library of anti-cancer antibodies since there is a possibility that other tumors can bear some of the same epitopes as the one that is being treated. The antibodies produced according to this method may be useful to treat cancerous disease in any number of patients who have cancers that bind to these antibodies.

Using substantially the process of U.S. Pat. No. 6,180,370, the mouse monoclonal antibody AR2A72.10 was obtained following immunization of mice with cells from a breast tumor biopsy. Within the context of this application, anti-cancer antibodies having either cell-killing (cytotoxic) or cell-growth inhibiting (cytostatic) properties will hereafter be referred to as cytotoxic. These antibodies can be used in aid of staging and diagnosis of a cancer, and can be used to treat tumor metastases. The AR2A72.10 antigen was expressed on the cell surface of a broad range of ovarian cancer cell lines, as well as normal skin and lung cell lines. The ovarian cancer cell lines ES-2, OV2008, C13, OCC-1, Ovca-429, OVCAR-3 and SKOV-3 were susceptible to the cytotoxic effects of AR2A72.10.

The results of AR2A72.10 cytotoxic activity against ovarian cancer cells in vitro was further extended by establishing its anti-tumor activity in vivo. In an in vivo model of human ovarian cancer, ES-2 ovarian cancer cells were implanted intraperitoneally in severe combined immunodeficient (SCID) mice, as they are incapable of rejecting the human tumor cells due to a lack of certain immune cells. Pre-clinical xenograft tumor models are considered valid predictors of therapeutic efficacy. The ovarian tumor cell line ES-2 has been evaluated as an in vivo xenograft model in immunodeficient mice. The successful engraftment or ‘take-rate’ of ES-2 tumors and the sensitivity of the tumors to standard chemotherapeutic agents have characterized them as suitable models of human cancer. In the studies described herein, the ES-2 cell line was stably transfected with a gene producing secreted alkaline phosphatase (SEAP), so that SEAP levels could be evaluated in mouse serum as a surrogate marker for tumor burden.

In the ES-2+SEAP model, the survival was significantly extended in the AR2A72.10 treatment group in comparison to the group treated with the buffer control (p<0.0001). A cohort of the animals receiving AR2A72.10 treatment had a decreased amount of circulating SEAP. Two mice demonstrated a reduction in circulating SEAP levels during the treatment period. One mouse exhibited complete elimination of circulating SEAP that extended past the treatment period. The absence of circulating SEAP in this one mouse correlated with the lack of any visible tumor upon necropsy on day 84. The results for these 3 animals suggest that response to antibody treatment can range from partial and temporary to complete elimination of circulating SEAP levels representative of tumor burden. In all, these results in which AR2A72.10 produced benefits (improved survival and decreased tumor burden in comparison to control treatment) in an ovarian model of human cancer suggest pharmacologic and pharmaceutical benefits of this antibody for cancer therapy in mammals, including man.

Localization of the AR2A72.10 antigen and its prevalence within ovarian cancer patients is important in assessing the benefits of immunotherapy to this group of patients. To address antigen expression in ovarian tumors from cancer patients, tumor tissue samples from 59 individual ovarian cancer patients were screened for expression of the AR2A72.10 antigen. From the 55 representative sections, 38% stained positively with AR2A72.10. Ovarian cancer is very heterogeneous in nature; cancers can derive from multiple cell types. This suggests that the antigenic phenotypes on different cancer types should also be heterogeneous. The staining pattern demonstrated that the antigen for AR2A72.10 was present in a significant proportion of human ovarian cancers, thus making it a suitable target for antibody therapy.

To further extend the potential therapeutic benefit of AR2A72.10, the frequency and localization of the antigen within various human cancer tissues was determined. Several cancer types, in addition to ovarian cancer, expressed the AR2A72.10 antigen. These included kidney (2/3), liver (2/3), parotid (2/3), uterus (2/4), nasal cavity, larynx, tongue, submandibular gland, pancreas, esophagus, gall bladder (all 1/1). Other tumor tissues were negative for AR2A72.10 antigen expression; these included breast (0/1), lymph node (0/2), brain (0/2), thyroid (0/2) and lung (0/3). As with human ovarian cancer tissue, AR2A72.10 staining was localized within the cytoplasm and on the membrane of cancerous cells. This data serves as evidence that the antigen is expressed in humans, and on multiple types of cancers. In toto, this data demonstrates that the AR2A72.10 antigen is a cancer associated antigen and is expressed in humans, and is a pathologically relevant cancer target. Further, this data also demonstrates the binding of AR2A72.10 antibody to human cancer tissues, and can be used appropriately for assays that can be diagnostic, predictive of therapy, or prognostic. In addition, the cell membrane localization of this antigen permits the use of this antigen, its gene or derivatives, its protein or its variants to be used for assays that can be diagnostic, predictive of therapy, or prognostic.

In all, this invention teaches the use of the AR2A72.10 antigen as a target for a therapeutic agent, that when administered can reduce the tumor burden of a cancer expressing the antigen in a mammal, and can also lead to a prolonged survival of the treated mammal. This invention also teaches the use of CDMAB (AR2A72.10), and its derivatives, to target its antigen to reduce the tumor burden of a cancer expressing the antigen in a mammal, and to prolong the survival of a mammal bearing tumors that express this antigen. Furthermore, this invention also teaches the use of detecting the AR2A72.10 antigen in cancerous cells that can be useful for the diagnosis, prediction of therapy, and prognosis of mammals bearing tumors that express this antigen.

If a patient is refractory to the initial course of therapy or metastases develop, the process of generating specific antibodies to the tumor can be repeated for re-treatment. Furthermore, the anti-cancer antibodies can be conjugated to red blood cells obtained from that patient and re-infused for treatment of metastases. There have been few effective treatments for metastatic cancer and metastases usually portend a poor outcome resulting in death. However, metastatic cancers are usually well vascularized and the delivery of anti-cancer antibodies by red blood cells can have the effect of concentrating the antibodies at the site of the tumor. Even prior to metastases, most cancer cells are dependent on the host's blood supply for their survival and anti-cancer antibodies conjugated to red blood cells can be effective against in situ tumors as well. Alternatively, the antibodies may be conjugated to other hematogenous cells, e.g. lymphocytes, macrophages, monocytes, natural killer cells, etc.

There are five classes of antibodies and each is associated with a function that is conferred by its heavy chain. It is generally thought that cancer cell killing by naked antibodies are mediated either through antibody-dependent cell-mediated cytotoxicity (ADCC) or complement-dependent cytotoxicity (CDC). For example murine IgM and IgG2a antibodies can activate human complement by binding the C-1 component of the complement system thereby activating the classical pathway of complement activation which can lead to tumor lysis. For human antibodies, the most effective complement-activating antibodies are generally IgM, IgG3 and IgG1. Murine antibodies of the IgG2a and IgG3 isotype are effective at recruiting cytotoxic cells that have Fc receptors which will lead to cell killing by monocytes, macrophages, granulocytes and certain lymphocytes. Human antibodies of both the IgG1 and IgG3 isotype mediate ADCC.

Another possible mechanism of antibody-mediated cancer killing may be through the use of antibodies that function to catalyze the hydrolysis of various chemical bonds in the cell membrane and its associated glycoproteins or glycolipids, so-called catalytic antibodies.

There are two additional mechanisms of antibody-mediated cancer cell killing which are more widely accepted. The first is the use of antibodies as a vaccine to induce the body to produce an immune response against the putative antigen that resides on the cancer cell. The second is the use of antibodies to target growth receptors and interfere with their function or to down regulate that receptor so that its function is effectively lost.

The clinical utility of a cancer drug is based on the benefit of the drug under an acceptable risk profile to the patient. In cancer therapy survival has generally been the most sought after benefit, however there are a number of other well-recognized benefits in addition to prolonging life. These other benefits, where treatment does not adversely affect survival, include symptom palliation, protection against adverse events, prolongation in time to recurrence or disease-free survival, and prolongation in time to progression. These criteria are generally accepted and regulatory bodies such as the U.S. Food and Drug Administration (F.D.A.) approve drugs that produce these benefits (Hirschfeld et al. Critical Reviews in Oncology/Hematolgy 42:137-143 2002). In addition to these criteria it is well recognized that there are other endpoints that may presage these types of benefits. In part, the accelerated approval process granted by the U.S. F.D.A. acknowledges that there are surrogates that will likely predict patient benefit. As of year-end (2003), there have been sixteen drugs approved under this process, and of these, four have gone on to full approval, i.e., follow-up studies have demonstrated direct patient benefit as predicted by surrogate endpoints. One important endpoint for determining drug effects in solid tumors is the assessment of tumor burden by measuring response to treatment (Therasse et al. Journal of the National Cancer Institute 92(3):205-216 2000). The clinical criteria (RECIST criteria) for such evaluation have been promulgated by Response Evaluation Criteria in Solid Tumors Working Group, a group of international experts in cancer. Drugs with a demonstrated effect on tumor burden, as shown by objective responses according to RECIST criteria, in comparison to the appropriate control group tend to, ultimately, produce direct patient benefit. In the pre-clinical setting tumor burden is generally more straightforward to assess and document. In that pre-clinical studies can be translated to the clinical setting, drugs that produce prolonged survival in pre-clinical models have the greatest anticipated clinical utility. Analogous to producing positive responses to clinical treatment, drugs that reduce tumor burden in the pre-clinical setting may also have significant direct impact on the disease. Although prolongation of survival is the most sought after clinical outcome from cancer drug treatment, there are other benefits that have clinical utility and it is clear that tumor burden reduction, which may correlate to a delay in disease progression, extended survival or both, can also lead to direct benefits and have clinical impact (Eckhardt et al. Developmental Therapeutics: Successes and Failures of Clinical Trial Designs of Targeted Compounds; ASCO Educational Book, 39^{th} Annual Meeting, 2003, pages 209-219).

Accordingly, it is an objective of the invention to utilize a method for producing cancerous disease modifying antibodies from cells derived from a particular individual which are cytotoxic with respect to cancer cells while simultaneously being relatively non-toxic to non-cancerous cells, in order to isolate hybridoma cell lines and the corresponding isolated monoclonal antibodies and antigen binding fragments thereof for which said hybridoma cell lines are encoded.

It is an additional objective of the invention to teach CDMAB and antigen binding fragments thereof.

It is a further objective of the instant invention to produce CDMAB whose cytotoxicity is mediated through ADCC.

It is yet an additional objective of the instant invention to produce CDMAB whose cytotoxicity is mediated through CDC.

It is still a further objective of the instant invention to produce CDMAB whose cytotoxicity is a function of their ability to catalyze hydrolysis of cellular chemical bonds.

A still further objective of the instant invention is to produce CDMAB which are useful in a binding assay for diagnosis, prognosis, and monitoring of cancer.

Other objects and advantages of this invention will become apparent from the following description wherein, by way of illustration and example, certain embodiments of this invention are set forth.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Representative FACS histograms of AR2A72.10 and anti-Fas (positive control) antibodies directed against several cancer and non-cancer cell lines.

FIG. 2. Kaplan Meier plot showing survival of mice treated with AR2A72.10 compared to the group treated with the buffer control.

FIG. 3. Effect of AR2A72.10 on secreted alkaline phosphatase in 3 mice compared to the control buffer-treated group.

FIG. 4. Representative micrographs showing the binding pattern obtained with AR2A72.10 on tissue sections from a human ovarian cancer tissue array demonstrating positive staining. A. Papillary serous cystadenocarcinoma; B. Dysgerminoma; and negative staining C. Mucinous cystadenoma. Magnification is 200×.

FIG. 5. Representative micrographs showing the binding of AR2A72.10 on tissue sections from a multiple human cancers tissue array demonstrating positive staining. A. pancreas adenocarcinoma; B. kidney renal cell carcinoma; and negative staining C. lung adenocarcinoma. Magnification is 200×.

### DETAILED DESCRIPTION OF THE INVENTION

### Example 1

### Hybridoma Production—Hybridoma Cell Line: AR2A72.10

The hybridoma cell line AR2A72.10 was deposited, in accordance with the Budapest Treaty, with the International Depository Authority of Canada (IDAC), Bureau of Microbiology, Health Canada, 1015 Arlington Street, Winnipeg, Manitoba, Canada, R3E 3R2, on Apr. 27, 2004, under Accession Number 270404-01. In accordance with 37 CFR 1.808, the depositors assure that all restrictions imposed on the availability to the public of the deposited materials will be irrevocably removed upon the granting of a patent.

To produce the hybridoma that produces the anti-cancer antibody AR2A72.10, a fresh single cell suspension of a breast patient's tumor that had been passaged as a solid tumor in SCID mice, was prepared in PBS. IMMUNEASY™ (Qiagen, Venlo, Netherlands) adjuvant was prepared for use by gentle mixing. Five to seven week old BALB/c mice were immunized by injecting intramuscularly, 3.3 million cells in 50 microliters of the antigen-adjuvant mixtures. Newly prepared antigen-adjuvant was used to boost the immunized mice intraperitoneally, 2 weeks after the initial immunization, with 3.3 million cells in 50 microliters. A spleen was used for fusion 3 days after the last immunization. The hybridomas were prepared by fusing the isolated splenocytes with NSO-1 myeloma partners. The supernatants from the fusions were tested during subcloning of the hybridomas. Isotyping of the supernatant from the hybridoma secreting AR2A72.10 confirmed the isotype of AR2A72.10 to be IgM, K.

After one round of limiting dilution hybridoma supernatants were tested for antibodies that demonstrated an anti-cancer effect in a cytotoxicity assay. Three human breast cancer cell lines were tested: MDA-MB-231, MDA-MB-468 and SKBR-3. The Live/Dead cytotoxicity assay was obtained from Molecular Probes (Eu, OR). The assays were performed according to the manufacturer's instructions with the changes outlined below. Cells were plated before the assay at the predetermined appropriate density. After 2 days, 100 mL of supernatant from the hybridoma microtitre plates were transferred to the cell plates and incubated in a 5 percent CO₂ incubator for 5 days. The wells that served as the positive controls were aspirated until empty and 100 μL of sodium azide (NaN₃) or cycloheximide was added. After 5 days of treatment, the plates were then emptied by inverting and blotting dry. Room temperature DPBS (Dulbecco's phosphate buffered saline) containing MgCl₂ and CaCl₂ was dispensed into each well from a multichannel squeeze bottle, tapped 3 times, emptied by inversion and then blotted dry. 50 μL of the fluorescent calcein dye diluted in DPBS containing MgCl₂ and CaCl₂ was added to each well and incubated at 37° C. in a 5% CO₂ incubator for 30 minutes. The plates were read in a Perkin-Elmer HTS7000 fluorescenceplate reader and the data was analyzed in Microsoft Excel. The results are tabulated in Table 1. The AR2A72.10 hybridoma produced specific cytotoxicity of 15,27 and 44 percent in MDA-MB-231, MDA-MB-468 and SKBR-3 cells respectively. The known non-specific cytotoxic agent cycloheximide produced cytotoxicity as expected given the limitations of in vitro biological assays. AR2A72.10 was able to induce cytotoxicity in all 3 breast cancer cell lines, with SKBR-3 cells being the most sensitive. AR2A72.10 showed greater specific cytotoxicity in all 3 breast cancer cell lines in comparison to the positive control cycloheximide.

TABLE 1
In Vitro Cytotoxicity of AR2A72.10

| | Cytotoxicity (%) | | | | | |
|---|---|---|---|---|---|---|
| | MDA-MB-231 | | MDA-MB-468 | | SKBR-3 | |
| | Average | CV | Average | CV | Average | CV |
| AR2A72.10 | 15 | 1 | 27 | 3 | 44 | 1 |
| Cycloheximide | 49 | 13 | 24 | 18 | 56 | 11 |

### Example 2

AR2A72.10 monoclonal antibody was produced by culturing the hybridomas in CL-1000 flasks (BD Biosciences, Oakville, ON) with collections and reseeding occurring twice/week. The antibodies were purified according to standard antibody purification procedures with Protein G Sepharose 4 Fast Flow (Amersham Biosciences, Baie d'Urfé, QC).

AR2A72.10 was compared to a number of both positive (anti-Fas (EOS9.1, IgM, kappa, 20 micrograms/mL, eBioscience, San Diego, Calif.), anti-EGFR(C225, IgG1, kappa, 5 microgram/mL, Cedarlane, Homby, ON), Cycloheximide (0.5 micromolar, Sigma, Oakville, ON), NaN₃ (0.1%, Sigma, Oakville, ON)) and negative (107.3 (anti-TNP, IgG1, kappa, 20 microgram/mL, BD Biosciences, Oakville, ON), IgG Buffer (2%), IgM buffer (2%)) controls in a cytotoxicity assay (Table 2). Antibodies were tested against a panel of ovarian cancer, and normal cell lines. Two ovarian cancer (OVCAR-3, Sk-OV-3) and non-cancer (CCD-27sk, Hs888.Lu) cell lines were obtained from the ATCC, Manassas, Va. A2780-cp, A2780-s, C-13, OV2008, ES-2, Hey, OCC-1, OVCA-429 were obtained from the Ottawa Regional Cancer Center (Ottawa, ON). The Live/Dead cytotoxicity assay was obtained from Molecular Probes (Eugene, Oreg.). The assays were performed according to the manufacturer's instructions with the changes outlined below. Cells were plated before the assay at the predetermined appropriate density. After 2 days, purified antibody or controls were diluted into media, and then 100 microliters were transferred to the cell plates and incubated in a 5 percent CO₂ incubator for 5 days. The plate was then emptied by inverting and blotted dry. Room temperature DPBS containing MgCl₂ and CaCl₂ was dispensed into each well from a multi-channel squeeze bottle, tapped three times, emptied by inversion and then blotted dry. 50 microliters of the fluorescent calcein dye diluted in DPBS containing MgCl₂ and CaCl₂ was added to each well and incubated at 37° C. in a 5 percent CO₂ incubator for 30 minutes. The plates were read in a Perkin-Elmer HTS7000 fluorescence plate reader and the data was analyzed in Microsoft Excel and the results were tabulated in Table 2. The data represented an average of four experiments tested in triplicate and presented qualitatively in the following fashion: 4/4 experiments greater than threshold cytotoxicity (+++), 3/4 experiments greater than threshold cytotoxicity (++), 2/4 experiments greater than threshold cytotoxicity (+). Unmarked cells in Table 1 represent inconsistent or effects less than the threshold cytotoxicity. The chemical cytotoxic agents induced their expected cytotoxicity while a number of other antibodies which were included for comparison also performed as expected given the limitations of biological cell assays. The AR2A72.10 antibody demonstrated cytotoxicity in multiple ovarian cancer cell lines, and was selective in its activity since not all cancer cell types were susceptible to antibody-mediated cytotoxicity. Furthermore, AR2A72.10 demonstrated functional specificity since it did not produce cytotoxicity against non-cancer cell types, which is important for the therapeutic utility of the antibody.

TABLE 2
In Vitro Cytotoxicity of Purified AR2A72.10

| | OVARIAN CANCER CELL LINES | | | | | | | | | | NORMAL | |
|---|---|---|---|---|---|---|---|---|---|---|---|---|
| Antibody | A2780-cp | A2780-s | C-13 | OV2008 | ES-2 | Hey | OCC-1 | OVCA-429 | OVCAR-3 | Sk-OV-3 | CCD 27sk | Hs888 Lu |
| AR2A72.10 | | | ++++ | ++ | ++ | | + | +++ | + | ++ | | |
| (20 μg/mL) | | | | | | | | | | | | |
| Positive Controls | | | | | | | | | | | | |
| anti-fas (10 μg/mL) | | ++++ | ++++ | + | | ++++ | ++++ | | ++++ | | | |
| anti-Her2/neu | | + | | | | | | | | | | |
| (10 μg/mL) | | | | | | | | | | | | |
| anti-EGFR | | | ++++ | | | | + | | + | ++ | +++ | |
| (C225, 5 μg/mL) | | | | | | | | | | | | |
| Cycloheximide | ++++ | ++++ | ++++ | ++++ | ++++ | ++++ | ++++ | ++++ | ++++ | ++++ | ++++ | ++++ |
| (0.5 μM) | | | | | | | | | | | | |
| NaN3 (0.1%) | ++++ | ++++ | ++++ | ++++ | ++++ | ++++ | ++++ | ++++ | ++++ | ++++ | | +++ |
| Negative Controls | | | | | | | | | | | | |
| AR3BD-26 | | | + | + | + | | | +++ | | | | |
| (1 gM, 20 μg/mL) | | | | | | | | | | | | |
| 1 gM Buffer (2%) | | | +++ | ++ | | | + | + | + | | | + |

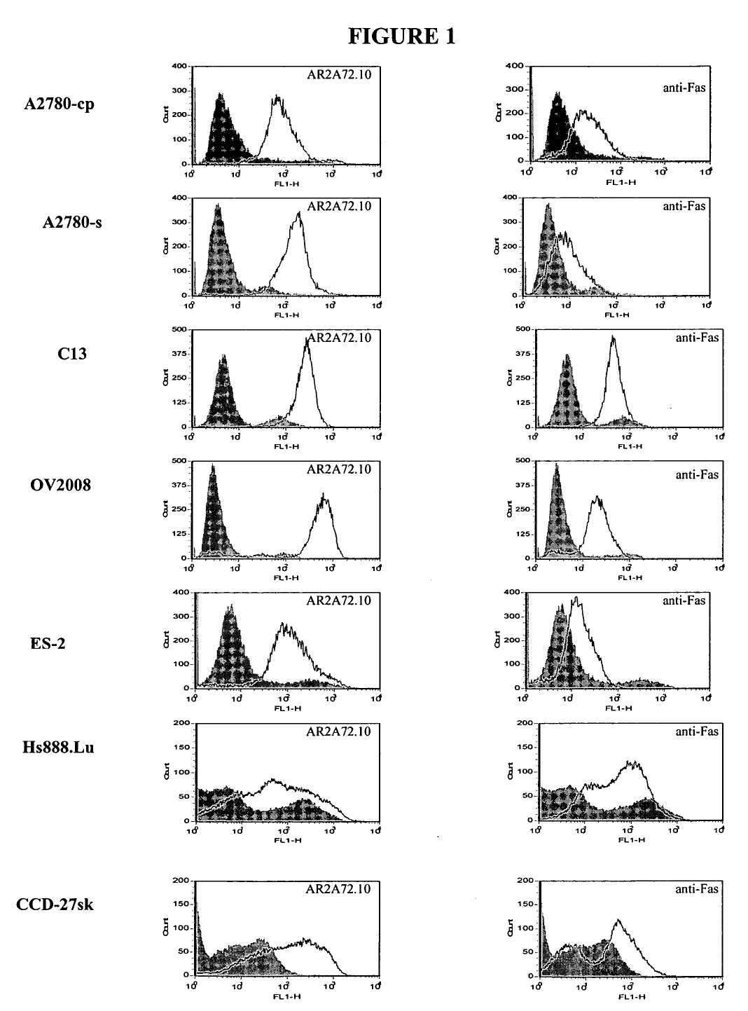

Binding of AR2A72.10 to the above-mentioned panel of cancer and normal cell lines was also assessed by flow cytometry (FACS). Cells were prepared for FACS by initially washing the cell monolayer with DPBS (without Ca⁺⁺ and Mg⁺⁺). Cell dissociation buffer (INVITROGEN, Burlington, ON) was then used to dislodge the cells from their cell culture plates at 37° C. After centrifugation and collection, the cells were resuspended in DPBS containing MgCl₂, CaCl₂ and 2 percent fetal bovine serum at 4° C. (staining media) and counted, aliquoted to appropriate cell density, spun down to pellet the cells and resuspended in staining media at 4° C. in the presence of test antibodies (AR2A72.10) or control antibodies (isotype control, anti-Fas) at 20 μg/mL on ice for 30 minutes. Prior to the addition of Alexa Fluor 488-conjugated secondary antibody the cells were washed once with staining media. The Alexa Fluor 488-conjugated antibody in staining media was then added for 30 minutes. The cells were then washed for the final time and resuspended in fixing media (staining media containing 1.5% paraformaldehyde). Flow cytometric acquisition of the cells was assessed by running samples on a FACScan using the CellQuest software (BD Biosciences, Oakville, ON). The forward (FSC) and side scatter (SSC) of the cells were set by adjusting the voltage and amplitude gains on the FSC and SSC detectors. The detectors for the fluorescence (FITC) channel was adjusted by running cells stained only with Alexa Fluor 488-conjugated secondary antibody such that cells had a uniform peak with a median fluorescent intensity of approximately 1-5 units. For each sample, approximately 10,000 stained fixed cells were acquired for analysis and the results are presented in FIG. 1. Table 3 tabulated the mean fluorescence intensity fold increase above isotype control and is presented qualitatively as: less than 1.5 (−); 1.5 to 3 (+); 4 to 10 (++); 11 to 100 (+++) and >100 (++++).

Representative histograms of AR2A72.10 antibody were compiled for FIG. 1. AR2A72.10 showed binding to both the cancerous and normal cell lines tested. Despite generally universal binding to these cells, AR2A72.10 did not produce uniform cytoxicity (see above). This was evidence that the degree of binding was not necessarily predictive of the outcome of antibody ligation of its cognate antigen, and was a non-obvious finding. This suggested that the context of antibody ligation in different cells was determinative of cytotoxicity rather than just antibody binding. It was noted that the binding of the antibody was higher in the parental lines of two of the cancer models (A2780-s and OV2008) compared to the cisplatin-resistant variant sublines (A2780-cp and C-13, respectively) derived from these models.

TABLE 3
FACS Analysis of AR2A72.10

| | OVARIAN CANCER CELL LINES | | | | | | | | | NORMAL | |
|---|---|---|---|---|---|---|---|---|---|---|---|
| Antibody | A2780-cp | A2780-s | C-13 | OV2008 | ES-2 | Hey | OCC-1 | OVCA-429 | OVCAR-3 | CCD-27sk | Hs888.Lu |
| AR2A72.10 | +++ | +++ | +++ | ++++ | +++ | +++ | +++ | +++ | +++ | +++ | + |
| (20 mg/mL) | | | | | | | | | | | |
| anti-fas (10 mg/mL) | ++ | + | ++ | ++ | + | ++ | +++ | + | ++ | ++ | +++ |
| anti-EGFR | + | +++ | +++ | +++ | +++ | +++ | +++ | +++ | +++ | ++ | +++ |
| (C225, 5 mg/mL) | | | | | | | | | | | |

### Example 3

### In Vivo ES-2+SEAP Established Tumor Experiment

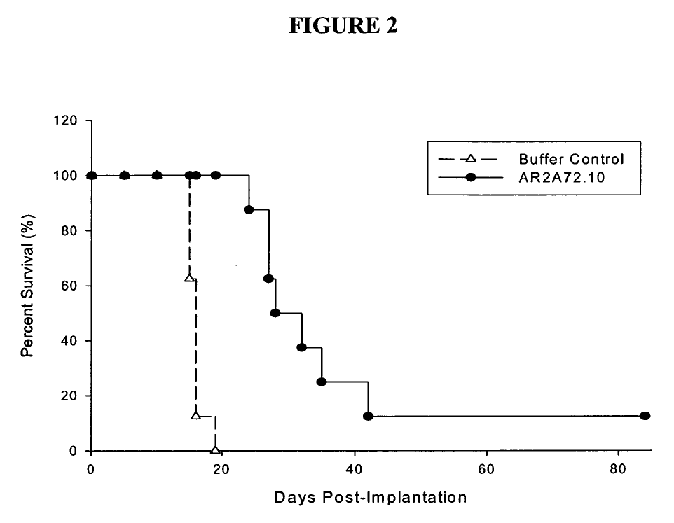

With reference to FIGS. 2 and 3, 6 to 8 week old female athymic nude mice were intraperitoneally implanted with 10 million ES-2+SEAP human ovarian cancer cells stably transfected to express human placental secreted alkaline phosphatase (SEAP). The 10 million ovarian cancer cells were resuspended in 500 microlitres serum-free (α-MEM. Tumor growth was confirmed with the sacrifice of 3 mice on day 7. Following the confirmation of tumor growth on day 7, 8 mice were randomized into each of 2 treatment groups. AR2A72.10 or buffer control was administered intraperitoneally with 10 mg/kg/dose at a volume of 250 microliters after dilution from the stock concentration with a diluent that contained 2.7 mM KCl, 1 mM KH₂PO₄, 137 mM NaCl and 20 mM Na₂HPO₄. The antibodies were then administered once per day for 5 doses and then once every other day for another 5 doses for a total of 10 doses. Tumor burden was extrapolated by measuring circulating SEAP levels and assessed visually upon necropsy at the termination of the study or when individual animals reached CCAC end-points. Body weights of the animals were recorded for the duration of the study. At the end of the study all animals were euthanised according to CCAC guidelines.

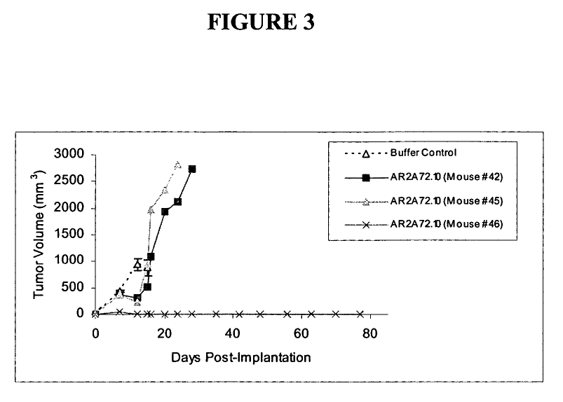

As shown in FIG. 2, survival was significantly extended in the AR2A72.10 treatment group in comparison to the group treated with the buffer control (p<0.0001) as determined with the log-rank test. circulating plasma SEAP levels (indicative of tumor burden) were taken at various times during the study for analysis. FIG. 3 displays the SEAP levels during the duration of the study for controls, and 3 AR2A72.10-treated mice. Due to intrinsic variability, there was no significant difference between the mean SEAP level of the AR2A72.10 and control-treated groups at the end of the treatment period. However, 3 animals receiving AR2A72.10 treatment had a decreased amount of circulating SEAP (FIG. 3). Two mice demonstrated a slight reduction in circulating SEAP levels during the treatment period. One mouse exhibited complete elimination of circulating SEAP that extended past the treatment period. The absence of circulating SEAP in this one mouse correlated with the lack of any visible tumor upon necropsy on day 84. The results for these 3 animals suggest that response to antibody treatment can range from partial and temporary to complete elimination of circulating SEAP levels representative of tumor burden. In all, these results in which AR2A72.10 produced benefits (improved survival and decreased tumor burden in comparison to control treatment) in an ovarian model of human cancer suggest pharmacologic and pharmaceutical benefits of this antibody for cancer therapy in mammals, including man.

### Example 4

### Human Ovarian Tumor Tissue Staining

An IHC study was undertaken to determine the cancer association of the AR2A72.10 antigen with human ovarian cancers and whether AR2A72.10 was likely to recognize human ovarian cancers. IHC optimization studies were performed initially in order to determine the conditions for further experiments. These optimization studies included the use of positive control (monoclonal mouse anti-A2B5 directed towards a ganglioside epitope expressed on membranes of neuroendocrine and glial cells; Dako, Toronto, Ontario) and negative IgM isotype control (directed towards Aspergillus niger glucose oxidase, an enzyme which is neither present nor inducible in mammalian tissues; Dako, Toronto, Ontario) antibodies, which performed as expected. The AR2A72.10 monoclonal antibody was produced and purified as stated above.

Tissue sections were deparaffinized by drying in an oven at 58° C. for 1 hour and dewaxed by immersing in xylene 5 times for 4 minutes each in Coplin jars. Following treatment through a series of graded ethanol washes (100%-75%) the sections were re-hydrated in water. The slides were immersed in 10 mM citrate buffer at pH 6 (Dako, Toronto, Ontario) then microwaved at high, medium, and low power settings for 5 minutes each and finally immersed in cold PBS. Slides were then immersed in 3% hydrogen peroxide solution for 6 minutes, washed with PBS three times for 5 minutes each, dried, and incubated with Universal blocking solution (Dako, Toronto, Ontario) for 5 minutes at room temperature. AR2A72.10 was diluted in antibody dilution buffer (Dako, Toronto, Ontario) to its working concentration (7 μg/mL for each antibody) and incubated overnight for 1 hour at room temperature. The slides were washed with PBS 3 times for 5 minutes each. Immunoreactivity of the primary antibodies was detected/visualized with HRP conjugated secondary antibodies as supplied (Dako Envision System, Toronto, Ontario) for 30 minutes at room temperature. Following this step the slides were washed with PBS 3 times for 5 minutes each and a color reaction developed by adding DAB (3,3′-diaminobenzidine tetrahydrachloride, Dako, Toronto, Ontario) chromogen substrate solution for immunoperoxidase staining for 10 minutes at room temperature. Washing the slides in tap water terminated the chromogenic reaction. Following counterstaining with Meyer's Hematoxylin (Sigma Diagnostics, Oakville, ON), the slides were dehydrated with graded ethanols (75-100%) and cleared with xylene. Using permanent mounting media (Dako Faramount, Toronto, Ontario) the slides were coverslipped. Slides were microscopically examined using an Axiovert 200 (Zeiss Canada, Toronto, ON) and digital images acquired and stored using Northern Eclipse Imaging Software (Mississauga, ON). Results were read, scored and interpreted by a pathologist.

An ovarian cancer tissue array derived from 59 ovarian cancer patients was used (Imgenex Corporation, San Diego, Calif.). The following information was provided for each patient: age and diagnosis.

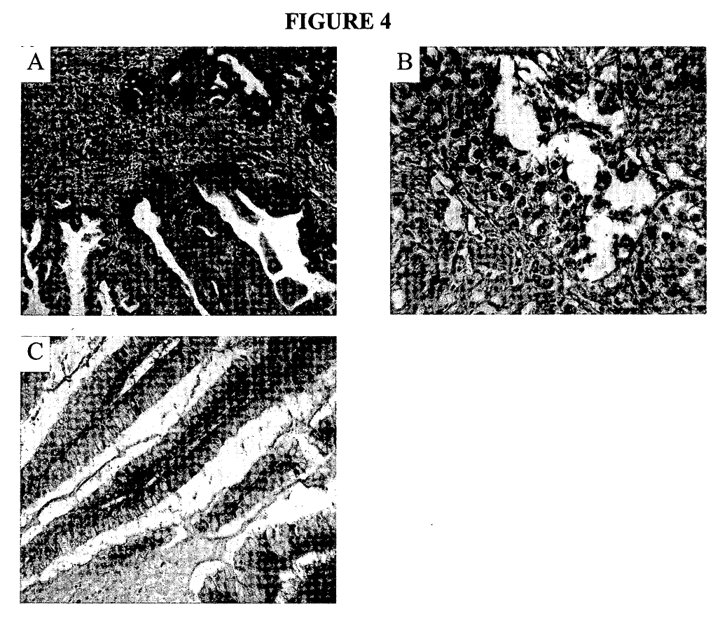

From the 55 representative sections, 38% stained positively with AR2A72.10 (Table 4). There appeared to be no correlation with cancer diagnosis; the highest staining sections were present in sections from a papillary serous cystadenocarcinoma and a dysgerminoma (FIG. 4). Ovarian cancer is very heterogeneous in nature; cancers can derive from multiple cell types. This suggests that the antigenic phenotypes on different cancer types should also be heterogeneous. The staining pattern demonstrated that the antigen for AR2A72.10 was present in a significant proportion of human ovarian cancers, thus making it a suitable target for antibody therapy.

TABLE 4
IHC On Human Ovarian Tumor Tissue

| | | | | | | Percent | |
|---|---|---|---|---|---|---|---|
| | | | Section | Tissue | Cellular | Stained | Staining |
| No. | Age | Diagnosis | Score | Specificity | Localization | Cells | Intensity |
| 1 | 65 | Serous papillary cystadenoma of borderline malignancy | The tumor section is totally necrotic (QC superbiochips) | | | | |
| 2 | 26 | mucinous Cystic tumor of borderline malignancy | + | Tumor cells | CMD | 50% | Weak |
| 3 | 35 | Serous papillary cystadenoma of borderline malignancy | +/− | Tumor cells | CMD | <50% | Equivocal |
| 4 | 65 | Serous cystadenocarcinoma | − | | | | Negative |
| 5 | 48 | Endometroid carcinoma | − | | | | Negative |
| 6 | 44 | Granulose cell tumor | − | | | | Negative |
| 7 | 49 | Endometroid carcinoma | − | | | | Negative |
| 8 | 67 | Mucinous cystadenoma of borderline malignacy | − | | | | Negative |
| 9 | 75 | Serous cystadenofibroma | − | | | | Negative |
| 10 | 50 | papillary serous cystadenocarcinoma, | − | | | | Negative |
| | | poorly differentiated | | | | | |
| 11 | 44 | Fibroma | − | | | | Negative |
| 12 | 50 | papillary serous cystadenocarcinoma | + | Tumor cells | CMD | <50% | Weak |
| 13 | 47 | Endometriod carcinoma | − | | | | Negative |
| 14 | 38 | mixed brenner tumor and mucinous cystadenoma | ++ | Tumor cells | CMD | <50% | Moderate |
| 15 | 58 | papillary serous cystadenocarcinoma | − | | | | Negative |
| 16 | 70 | papillary serous cystadenocarcinoma | + | Tumor cells | CMD | <50% | Moderate |
| 17 | 65 | stroma ovarii | − | Tumor cells | | | Negative |
| | | | | Stroma | | | Strong |
| 18 | 79 | Endometrioid adenocanthofibroma | ++ | Tumor cells | CMD | >50% | Weak-Moderate |
| 19 | 57 | serous surface papillary carcinoma | − | Tumor cells | | | Negative |
| | | | | SMF of | | | Weak |
| | | | | blood vessels | | | |
| 20 | 43 | papillary serous cystadenocarcinoma | − | | | | Negative |
| 21 | 68 | undiffrentiated carcinoma | + | Tumor cells | CMD | <50% | Weak |
| 22 | 50 | clear cell carcinoma | + | Tumor cells | CMD | <50% | Weak |
| 23 | 23 | dysgerminoma | − | | | | Negative |
| 24 | 51 | common epithelial carcinoma, poorly differentiated | + | Tumor cells | CMD | <50% | Weak |
| 25 | 70 | malignant mullerian mixed tumor | − | | | | Negative |
| 26 | 21 | dysgerminoma | ++ | Tumor cells | CMD | <10% | Moderate |
| 27 | 34 | fibrothecoma | − | | | | Negative |
| 28 | 61 | papillary serous cystadenocarcinoma | +/− | Tumor cells | CMD | <10% | Equivocal |
| 29 | 54 | sertoli-leydig cell tumor | − | | | | Negative |
| 30 | 53 | fibrothecoma | − | | | | Negative |
| 31 | 54 | fibrothecoma | +/− | Tumor cells | CMD | <10% | Equivocal |
| 32 | 34 | papillary serous cystadenocarcinoma | +++ | Tumor cells | CMD | >50% | Moderate-Strong |
| 33 | 31 | dysgerminoma | +/− | Tumor cells | CMD | <10% | Equivocal |
| 34 | 51 | papillary serous cystadenocarcinoma | + | Tumor cells | CMD | 50% | Weak |
| 35 | 61 | granulosa-theca cell tumor | + | Tumor cells | CMD | <10% | Weak |
| | | | | Necrotic area | | | Strong |
| 36 | 24 | sertoli-leydig cell tumor | − | | | | Negative |
| 37 | 27 | sclerosing stromal tumor | + | Tumor cells | CMD | >50% | Weak |
| 38 | 41 | clear cell carcinoma | − | | | | Negative |
| 39 | 56 | malignant mullerian mixed tumor | − | | | | Negative |
| 40 | 41 | clear cell carcinoma | + | Tumor cells | CMD | <50% | Weak |
| 41 | 27 | embryonal carcinoma | The tumor section is totally necrotic (QC superbiochips) | | | | |
| 42 | 55 | granulosa cell tumor | + | Tumor cells | CMD | >50% | Weak |
| 43 | 23 | dysgerminoma | +++ | Tumor cells | CMD | >50% | Strong |
| 44 | 64 | papillary serous cystadenocarcinoma | + | Tumor cells | CMD | <50% | Weak |
| 45 | 43 | metastatic adenocarcinoma | − | | | | Negative |
| 46 | 48 | malignant lymphoma | − | | | | Negative |
| 47 | 50 | adenocarcinoma from salpinx | The section is not representative (QS superbiochips) | | | | |
| 48 | 25 | fibrothecoma | − | | | | Negative |
| 49 | 46 | metastatic undiffefentiated carcinoma | − | | | | Negative |
| 50 | 37 | Metastatic signet ring carcinoma (krukenberg tumor) | − | | | | Negative |
| 51 | 55 | Serous cystadenocarcinoma, moderatly diffrentiated | − | | | | Negative |
| 52 | 55 | fibrothecoma | − | | | | Negative |
| 53 | 45 | Serous cystadenocarcinoma, moderatly diffrentiated | The section is not representative (QC superbiochips) | | | | |
| 54 | 37 | endometrioid carcinoma | − | | | | Negative |
| 55 | 58 | fibrothecoma | − | | | | Negative |
| 56 | 65 | adenocarcinoma, poorly diffrentiated | − | | | | Negative |
| 57 | 39 | clear cell carcinoma | − | | | | Negative |
| 58 | 70 | fibroma | − | | | | Negative |
| 59 | 58 | clear cell carcinoma | − | | | | Negative |

Abbreviations:
C: cytoplasmic,
M: membranous,
D: diffuse

### Example 5

### Human Tumor Tissue Staining

An IHC study was undertaken to determine the cancer association of the AR2A72.10 antigen with a variety of human cancers. A various tumors tissue array derived from 60 cancer patients was used (Imgenex Corporation, San Diego, Calif.). The following information was provided for each patient: age, sex and diagnosis. The procedure for IHC from Example 4 was followed, except that all antibodies were used at a working concentration of 6 μg/mL.

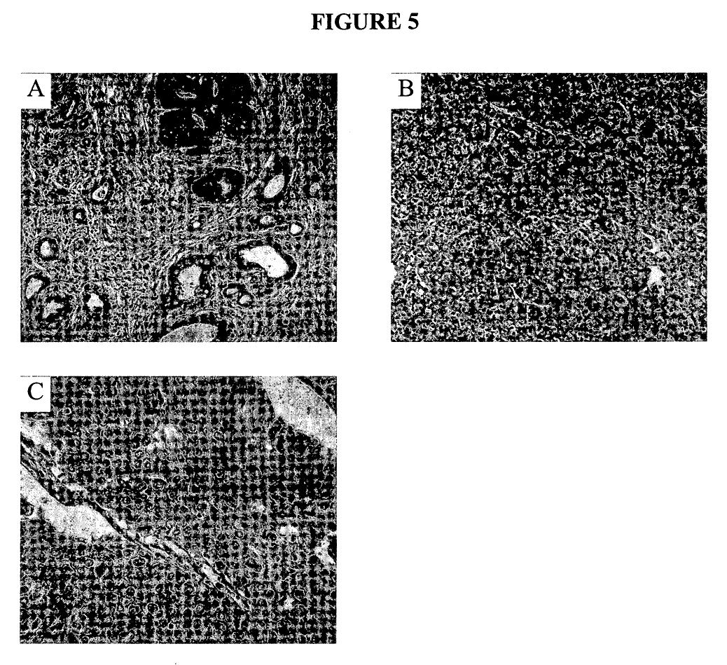

As outlined in Table 5, AR2A72.10 stained a number of various human cancers besides ovarian. The following tumor types were positive for AR2A72.10: kidney (2/3), liver (2/3), parotid (2/3), uterus (2/4), nasal cavity, larynx, tongue, submandibular gland, pancreas, esophagus, gall bladder (all 1/1) (FIG. 5). Several other tumor types also occasionally stained positive. Other tumor tissues were negative for AR2A72.10 antigen expression; these included breast (0/1), lymph node (0/2), brain (0/2), thyroid (0/2) and lung (0/3). As seen with the ovarian cancers, AR2A72.10 staining was localized within the cytoplasm and on the membrane of cancerous cells.

Therefore, it appears that the AR2A72.10 antigen is not solely found on the membranes of breast cancers but also on the membrane of a large variety of tumor types. These results indicate that AR2A72.10 has potential as a therapeutic drug in a wider variety of tumor types in addition to ovarian cancer.

TABLE 5
IHC On Human Tumor Tissue

| | | | | | | | | Percent | |
|---|---|---|---|---|---|---|---|---|---|
| | | | | | Section | Tissue | Cellular | Stained | Staining |
| No. | Age | Sex | Organ | Diagnosis | Score | specificity | Localization | Cells | Intensity |
| 1 | 59 | M | Skin | Malignant Melanoma | − | | | | Negative |
| 2 | 25 | F | Skin | SSC | ++ | Tumor cells & Keratin | CMD | >50% | Modearte |
| 3 | 50 | F | Breast | Inf.D.Ca. | − | | | | Negative |
| 4 | 57 | F | Breast | Inv.Papillary.Ca. | NR | No tumor cells | | | |
| 5 | 35 | F | Breast | Inf.L.Ca | PD | | | | |
| 6 | 40 | M | Lymph node | Malignant Lymphoma, | − | | | | Negative |
| | | | | Immunoplastic | | | | | |
| 7 | 58 | M | Lymph node | Metastatic adenoca | − | | | | Negative |
| | | | | from stomach | | | | | |
| 8 | 53 | F | Bone | Osteosarcoma | − | | | | Negative |
| 9 | 26 | M | Bone | Giant cell tumor | + | Tumor cells | CMD | >50% | Weak-Moderate |
| 10 | 40 | M | bone | Chondrosarcoma | − | | | | Negative |
| 11 | 51 | F | Soft tissue | liposarcoma | − | | | | Negative |
| 12 | 47 | F | Soft tissue | Neurofibromatosis | − | Tumor cells | | | Negative |
| | | | | | | SMF of blood vessels | | | Weak |
| 13 | 74 | M | Nasal cavity | Inverted papilloma | + | Tumor cells | CMD | >50% | Weak |
| 14 | 57 | M | larynx | SCC | + | Tumor cells | CMD | >50% | Weak |
| 15 | 60 | M | lung | Adenocarcinoma | − | | | | Negative |
| 16 | 51 | F | lung | SCC | − | | | | Negative |
| 17 | 68 | F | lung | Adenocarcinoma | NR | Normal Lung only | | | |
| 18 | 60 | M | lung | Small cell Carcinoma | − | | | | Negative |
| 19 | 88 | F | tongue | SCC | ++ | Tumor cells | CMD | >50% | Weak-Moderate |
| 20 | 34 | F | Parotid tumor | Pleomorphic adenoma | +++ | Tumor cells | CMD | >50% | Strong |
| | | | | | | (Cartilage only) | | | |
| 21 | 50 | F | Parotid tumor | Warthin tumor | − | | | | Negative |
| 22 | 40 | F | Parotid tumor | Pleomorphic adenoma | + | Tumor cells | CMD | <50% | Weak |
| 23 | 56 | M | Submandibular | Salivary duct carcinoma | ++ | Tumor cells | CMD | >50% | Moderate |
| | | | gland | | | | | | |
| 24 | 69 | F | Liver | Cholangiocarcinoma | + | Tumor cells | CMD | >50% | Weak |
| 25 | 51 | M | Liver | Metastatic gastric Ca. | − | | | | Negative |
| 26 | 64 | M | Liver | HCC | +++ | Tumor cells | CMD | >50% | Strong |
| 27 | 62 | F | Gall bladder | Adenocarcinoma | ++ | Tumor cells | CMD | >50% | Moderate |
| 28 | 64 | F | pancreas | Adenocarcinoma | +++ | Tumor cells | CMD | >50% | Moderate-Strong |
| 29 | 68 | M | esophagus | SCC | +++ | Tumor cells | CMD | >50% | Moderate-Strong |
| 30 | 73 | M | stomach | Adenocarcinoma Poorly diff. | − | | | | Negative |
| 31 | 63 | M | stomach | Adenocarcinoma moderately | + | Tumor cells | CMD | >50% | Weak |
| | | | | diff. | | | | | |
| 32 | 59 | F | stomach | Signet ring cell carcinoma | − | | | | Negative |
| 33 | 62 | M | stomach | Malignant lymphoma | − | | | | Negative |
| 34 | 51 | M | stomach | Borderline stromal tumor | − | | | | Negative |
| 35 | 42 | M | Small intestine | Malignant stromal tumor | − | | | | Negative |
| 36 | 52 | F | appendix | Pseuomyxoma peritonia | − | | | | Negative |
| 37 | 53 | M | colon | Adenocarcinoma | − | | | | Negative |
| 38 | 67 | M | rectum | Adenocarcinoma | NR | No tumor cells | | | |
| 39 | 75 | F | kidney | Transitional Cell carcinoma | − | | | | Negative |
| 40 | 54 | F | kidney | Renal cell carcinoma | +++ | Tumor cells | CMD | >50% | Moderate-Strong |
| 41 | 75 | F | kidney | Renal cell carcinoma | + | Tumor cells | CMD | <50% | Weak |
| 42 | 65 | M | Urinary bladder | Poorly diff, carcinoma | − | | | | Negative |
| 43 | 67 | M | Urinary bladder | Transitional Cell carcinoma, | + | Tumor cells | CMD | >50% | Weak |
| | | | | High grade | | | | | |
| 44 | 62 | M | prostate | adenocarcinoma | NR | Completely | | | |
| | | | | | | haemorrhagic | | | |
| 45 | 30 | M | testis | Seminoma | − | | | | Negative |
| 46 | 68 | F | uterus | Endometrial adenocarcinoma | − | | | | Negative |
| 47 | 57 | F | uterus | Leimyosacoma | − | | | | Negative |
| 48 | 45 | F | uterus | Leiomyoma | ++ | Tumor cells | CMD | >50% | Moderate |
| 49 | 63 | F | Uterine cervix | SCC | ++ | Tumor cells | CMD | >50% | Weak-Moderate |
| 50 | 12 | F | ovary | Endodermal sinus tumor | NR | Completely | | | |
| | | | | | | haemorrhagic | | | |
| 51 | 33 | F | ovary | Mucinous adenocarcinoma | − | | | | Negative |
| 52 | 70 | F | ovary | Fibrothecoma | − | | | | Negative |
| 53 | 67 | F | Adrenal gland | Cortical carcinma | − | | | | Negative |
| 54 | 61 | F | Adrenal gland | pheohromcytoma | + | Tumor cells | CMD | >50% | Weak-Moderate |
| 55 | 54 | M | thyroid | Papillary carcinoma | − | | | | Negative |
| 56 | 58 | F | thyroid | Minimally Inv. | − | | | | Negative |
| | | | | Follicular carcinoma | | | | | |
| 57 | 74 | M | thymus | Thymoma | − | | | | Negative |
| 58 | 66 | F | brain | Meningioma | − | | | | Negative |
| 59 | 62 | M | brain | Glioblastoma multiforme | − | | | | Negative |

| | | | | | |
|---|---|---|---|---|---|
| 60 | 64 | F | Lymph node | Malignant Melanoma | Originally pigmented |

Abbreviations:
SMF: smooth muscle fiber,
C: cytoplasmic,
M: membranous,
D: diffuse,
G: granular,
PD: partially detached,
F: folded,
CD: completely detached.

In toto, AR2A72.10 is significantly more effective than buffer control in suppressing tumor growth and extending survival in an established tumor xenograft model of ovarian cancer in SCID mice. The antigen for AR2A72.10 is a relevant cancer target, as it is expressed on 38% of ovarian cancer sections from human patients and is also expressed on other human cancers, including kidney, liver and pancreas.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objects and obtain the ends and advantages mentioned, as well as those inherent therein. Any oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method of extending survival and/or delaying disease progression by treating a human tumor in a mammal, wherein said human tumor expresses an antigen which specifically binds to a monoclonal antibody which has the identifying characteristics of a monoclonal antibody encoded by a clone deposited with the IDAC as accession number 270404-01 or an antigen binding fragment thereof, comprising administering to said mammal said monoclonal antibody or an antigen binding fragment thereof in an amount effective to reduce said mammal's tumor burden, whereby disease progression is delayed and/or survival is extended.

2. The method of claim 1 wherein said monoclonal antibody is conjugated to a cytotoxic moiety.

3. The method of claim 2 wherein said cytotoxic moiety is a radioactive isotope.

4. The method of claim 1 wherein said monoclonal antibody activates complement.

5. The method of claim 1 wherein said monoclonal antibody mediates antibody dependent cellular cytotoxicity.

6. The method of claim 1 wherein said monoclonal antibody is a humanized antibody.

7. The method of claim 1 wherein said monoclonal antibody is a chimerized antibody.

8. An isolated monoclonal antibody encoded by the clone deposited with the IDAC as 270404-01.

9. The antibody of claim 8, which is a humanized antibody.

10. The antibody of claim 8, which is a chimerized antibody.

11. Antigen binding fragments of the isolated monoclonal antibody of claim 8.

12. Antigen binding fragments of the humanized antibody of claim 9.

13. Antigen binding fragments of the chimerized antibody of claim 10.

14. The isolated monoclonal antibody or antigen binding fragments of any one of claims 8, 9, 10, 11, 12 or 13 conjugated with a member selected from the group consisting of cytotoxic moieties, enzymes, radioactive compounds, and hematogenous cells;
whereby antibody conjugates are formed.

15. The isolated clone deposited with the IDAC as 270404-01.

16. A binding assay to determine presence of cancerous cells in a tissue sample selected from a human tumor comprising:
providing a tissue sample from said human tumor;
providing an isolated monoclonal antibody encoded by the clone deposited with the IDAC as 270404-01, or an antigen binding fragment thereof, or an antibody conjugate thereof;
contacting said isolated monoclonal antibody or antigen binding fragment thereof or antibody conjugate thereof with said tissue sample; and
determining binding of said isolated monoclonal antibody or antigen binding fragment thereof or antibody conjugate thereof with said tissue sample;
whereby the presence of said cancerous cells in said tissue sample is indicated.

17. The binding assay of claim 16 wherein the human tumor tissue sample is obtained from a tumor originating in a tissue selected from the group consisting of pancreatic tissue and ovarian tissue.

18. A process of isolating or screening for cancerous cells in a tissue sample selected from a human tumor comprising:
providing a tissue sample from said human tumor;
providing an isolated monoclonal antibody encoded by the clone deposited with the IDAC as 270404-01, or an antigen binding fragment thereof, or an antibody conjugate thereof;
contacting said isolated monoclonal antibody or antigen binding fragment thereof or antibody conjugate thereof with said tissue sample; and
determining binding of said isolated monoclonal antibody or antigen binding fragment thereof or antibody conjugate thereof with said tissue sample;
whereby said cancerous cells are isolated by said binding and their presence in said tissue sample is confirmed.

19. The process of claim 18 wherein the human tumor tissue sample is obtained from a tumor originating in a tissue selected from the group consisting of pancreatic tissue and ovarian tissue.
